(19) United States
(12) Patent Application Publication
(10) Pub. No.: US 2021/0210214 A1
(43) Pub. Date: Jul. 8, 2021

(54) OPTIMISATION SYSTEM AND METHOD

(71) Applicant: IMPERIAL COLLEGE OF SCIENCE, TECHNOLOGY AND MEDICINE, London Greater London (GB)

(72) Inventors: Adam Hampshire, London Greater London (GB); Robert Leech, London Greater London (GB)

(73) Assignee: IMPERIAL COLLEGE OF SCIENCE, TECHNOLOGY AND MEDICINE, London Greater London (GB)

(21) Appl. No.: 17/058,797

(22) PCT Filed: May 31, 2019
(86) PCT No.: PCT/GB2019/051513

§ 371 Date: Nov. 25, 2020

(30) Foreign Application Priority Data
May 31, 2018 (GB) 1808878.1

(51) Int. Cl. G16H 50/70; A61B 5/16; A61B 5/00

(52) U.S. Cl. CPC G16H 50/70; A61B 5/162; A61B 5/7275; A61B 5/7246; A61B 5/4088

(57) ABSTRACT

A computer-implemented method, a computer program and a system for optimising measurement processes are disclosed. The method, computer program and system comprise receiving one or more parameters for configuring a plurality of measurement processes, receiving one or more results, generating a predictor, determining updated parameters using the predictor, receiving one or more further results, generating an updated predictor and determining one or more enhanced parameters.

### FIELD OF THE INVENTION

The present invention relates to optimising measurement processes.

### BACKGROUND

Measurement processes are used in many fields to measure properties of systems, objects, organisms, materials and chemicals. Most measurement processes are suboptimal. They are influenced by a plethora of confounding factors casting doubt on their results and usefulness. In addition, many are unnecessarily expensive and/or destructive.

Much of the data collected by measurement processes is also redundant, or at least is not the most informative data that could have been collected. Likewise, the information collected is not fully exploited.

Neurocognitive functioning measurement processes are measurement processes for assessing neurocognitive function. Deriving useful data from such measurement processes is difficult due to the vast number of confounding factors. Furthermore, as neurocognitive function measurement processes require human subjects, they are expensive and time consuming to perform.

### SUMMARY

A first aspect of the specification provides a computer implemented method for optimising measurement processes, the method comprising:
receiving one or more parameters for configuring a plurality of measurement processes, wherein the plurality of measurement processes is configured to measure one or more latent variables;
receiving one or more results, wherein each of the results is obtained using at least one of the plurality of measurement processes and the at least one of the plurality of measurement processes are configured using at least one of the received parameters;
generating a predictor, wherein the predictor is usable to provide an estimate of the value of an objective function for a first plurality of unsampled parameter values and is usable to provide an uncertainty value for each of the plurality of unsampled parameter values, wherein generating the predictor comprises:
   determining the value of the objective function for the one or more parameters in dependence on the one or more results;
determining one or more updated parameters using the predictor;
receiving one or more further results, wherein each of the further results is obtained using at least one of the plurality of measurement processes and the at least one of the plurality of measurement processes are configured using at least one of the updated one or more parameters;
generating an updated predictor, wherein the updated predictor provides an estimate of the value of an objective function for a second plurality of unsampled parameter values and provides an uncertainty value for each of the plurality of unsampled parameter values, wherein generating the updated predictor comprises:
   determining the value of the objective function for the one or more updated parameters in dependence on the one or more further results;
determining one or more enhanced parameters using the updated predictor, wherein the value of the objective function for the one or more parameters in dependence on one or more enhanced results obtained when the measurement processes are configured using the one or more enhanced parameters, is greater than or less than, dependent on the definition of the objective function, the value of the objective function for the one or more parameters.

The objective function may be a measure of the precision of the measurements of the one or more latent variables provided by the plurality of measurement processes.

The method may further comprise storing the one or more enhanced parameters.

The method may further comprise performing at least one of the plurality of measurement processes. The measurement processes being configured using the one or more enhanced parameters.

The method may further comprise executing one or more computer programs for performing the plurality of measurement processes. The method may further comprise setting one or more variables of the one or more computer programs based on the one or more parameters. The method may further comprise setting the one or more variables of the one or more computer programs based on the one or more updated parameters. The results and further results received in the method may be received from the one or more computer programs.

At least one of the plurality of measurement processes may comprise measuring the reaction time of a subject. At least one of the plurality of measurement processes may comprise measuring the number of successful recognitions of a sequence. At least one of the plurality of measurement processes may comprise measuring the total time that a user takes to complete a given measurement process.

The one or more latent variables may measure quantifiable aspects of neurocognitive function.

Each of the aspects of neurocognitive function may be associated with one or more brain networks and/or one or more brain regions.

The acquisition function may be an expected improvement function

Determining the predictor may comprise performing Gaussian progress regression. Determining the updated predictor may comprise performing Gaussian process regression.

Determining the value of the objective function may comprise determining the difference between a cross-correlation matrix for the results and a target cross-correlation matrix.

The plurality of measurement processes may be selected from a greater plurality of measurement processes. The method may further comprise selecting the plurality of measurement processes from the greater plurality of measurement processes. Selecting the plurality of measurement processes may comprise receiving initial results for the greater plurality of measurement processes and identifying a subset of the greater plurality of measurement processes based on the received initial results. The identified subset may be adapted for discretising the measurements of the latent variables. Identifying the subset may comprise performing factor analysis on the received initial results.

A second aspect of the specification provides a computer program comprising instructions which, when the program is executed by a computer, cause the computer to carry out any method above.

A third aspect of the specification provides a computer-readable medium comprising instructions which, when executed by a computer, cause the computer to carry out any method above.

A fourth aspect of the specification provides a computer system comprising one or more processors operatively coupled to one or more memories, wherein the one or more memories store executable instructions which, when executed by the one or more processors, cause the computer system to carry out any method above.

A fifth aspect of the specifications provides a plurality of optimised measurement processes configured using one or more enhanced parameters determined according to any of the methods above. The plurality of optimised measurement processes may be performed by executing one or more computer programs.

### BRIEF DESCRIPTION OF THE DRAWINGS

Certain embodiments of the present invention will now be described, by way of example, with reference to the accompany drawings, in which:

FIG. 1 is a schematic block diagram of a system for determining enhanced measurement process parameters;

FIG. 2 is a flow diagram of an example method for determining enhanced measurement process parameters;

FIG. 3A illustrates results of a first iteration of a one-dimensional Bayesian optimisation;

FIG. 3B illustrates results of a second iteration of a one-dimensional Bayesian optimisation;

FIG. 3C illustrates results of a third iteration of a one-dimensional Bayesian optimisation;

FIG. 4A illustrates results of a first iteration of a two-dimensional Bayesian optimisation;

FIG. 4B illustrates results of a first iteration of a two-dimensional Bayesian optimisation;

FIG. 4C illustrates results of a second iteration of a two-dimensional Bayesian optimisation;

FIG. 5 illustrates a graphical user interface for a first measurement process;

FIG. 6A illustrates a graphical user interface for a second measurement process prior to a user interaction;

FIG. 6B illustrates a graphical user interface for a second measurement process after the user interaction;

FIG. 7 illustrates regions of the left cerebral hemisphere of the brain associated with neurocognitive functioning; and

FIG. 8 is a schematic diagram of a computer system.

### DETAILED DESCRIPTION OF THE CERTAIN EMBODIMENTS

### General Overview

For ease of explanation, an example of a system for determining enhanced measurement process parameters is described in the context of measurement processes for aspects of neurocognitive function. However, the system and processes herein described are applicable in many other contexts. Examples of such contexts include optimising processes for measuring quantifiable properties of: machinery, electronics, buildings, materials, and chemicals.

Sets of measurement processes (herein also referred to as “test batteries”) are used in many fields to measure properties of systems, objects, organisms, materials and chemicals. In many circumstances, these sets of measurement processes do not directly measure the properties in which the user of the measurement processes is interested. Instead, unobserved properties, known as latent, or hidden, variables, interest the user.

Latent variables are not measured directly either because they can be difficult, even impossible, to measure in an unmixed form, or because direct measurement requires destructive testing. Each observed property relates to several latent variables. Similarly, each latent variable is influenced by a number of observed properties. Quantitative models are used to derive measures of the latent variables from the observed properties.

Neurocognitive measurement processes aim to measure one or more underlying aspects of neurocognitive function. For example, they aim to measure subjects' attention, spatial visualisation ability, memory, verbal processing, reasoning and planning. Many existing measurement processes claim to measure a single aspect of neurocognitive function. In reality, these measurement processes relate to several underlying aspects of neurocognitive function. For example, a measurement process requiring a subject to click when a given pattern is presented to them will measure their ability to sustain attention, control motor outputs and process visual inputs.

The present invention seeks to provide system(s) and method(s) for optimising sets of these neurocognitive function measurement processes. For example, the neurocognitive measurement processes may be optimised to increase the precision and/or accuracy of their measurements of the underlying aspects of neurocognitive function. A high-level description of a system for optimising neurocognitive function measurement processes now follows.

Quantitative models of how underlying aspects of neurocognitive function are related to the results of a set of neurocognitive function measurement processes can be obtained using latent variable analysis. Suitable methods of latent variable analysis include hierarchical clustering, factor analysis, principle component analysis and independent component analysis. These quantitative models can then be used to produce measures of the underlying aspects of neurocognitive function, the latent variables. However, the set of measurement processes will not be optimised for this purpose. By modifying the design of the measurement processes, as set out below, the measurement processes of these latent variables can be optimised.

An objective function quantifies a property of the plurality of the measurement processes, e.g. the precision of their measurements of the latent variables. Lower or higher values of the objective function may be desired, according to the definition of the objective function, and optimisation will respectively aim to minimise or maximise the objective function. For example, a lower or higher value of the objective function may be indicative of the plurality of measurement processes providing more precise measurements of the latent variables, and optimisation will respectively aim to minimise or maximise the objective function. For clarity, and as it is more typical, lower objective function values are regarded as preferable and, as such, hereinafter reference will be made to minimising the objective function. All such references, however, should be understood as encompassing maximising the objective function when higher values are desired.

Each of the measurement processes is configurable using one or more parameters. For example, the parameters for a measurement process requiring a subject to click when a given pattern is displayed could be the length of time each pattern is displayed for, and the number of items or complexity in each pattern. Varying the parameters affects how much the result of the measurement process is influenced by a specific aspect of cognition. As an example, decreasing the time each pattern is displayed for increases the extent to which the results of the example measurement process are affected by subjects' attention and motor control. Similarly, increasing the number of items in each pattern increases the extent to which they are affected by subjects' visual processing abilities.

The parameters of the measurement processes are set to initial values. These initial values may be random, user-provided, retrieved from a data store or quantitatively derived. One or more results are then collected using the measurement processes.

A parameter optimiser receives the parameters and associated results. Using these, the parameter optimiser derives a predictor. For each set of parameter values, the predictor provides a mean estimate of the objective function and an uncertainty value indicating the degree of certainty in its estimate, e.g. the variance. For example, the prediction of the objective function at sampled parameter values are relatively certain while the predictions far from any sampled points are substantially less certain. The predictor may be obtained using Gaussian process regression or Student-t process regression.

An acquisition function is then applied to the predictor to provide a so-called “usefulness” for all sets of parameters within a given parameter space, whereby a value, such as a maximum or a minimum, corresponding to the knowledge gainable from sampling a given parameter set is provided. The acquisition function determines usefulness based on both the mean estimate and uncertainty value provided by the predictor. To illustrate, parameters for which the predictor gives a mean estimate slightly greater than the minimal mean estimate but with a relatively high uncertainty, compared to the uncertainty for other parameter sets, would have a high usefulness. It is reasonably likely that the true objective function value for these parameters is lower than that of the current minimal mean estimate. Correspondingly, parameters for which the predictor gives a high mean estimate and a relatively low uncertainty would have a low usefulness, as it is very unlikely that the true objective function value for these parameters is less than the current minima.

Different acquisition functions give different weights to the mean estimate and the variance. This is known as the trade-off between “exploitation” and “exploration”. Giving more weight to the mean estimate finds a ‘good’ value, e.g. a local minimum, that can be exploited more quickly, but potentially at the expense of missing a global optimum far away in the sample space. Giving more weight to the uncertainty can help to explore more parameter sets, but potentially means that adequate values, such as local minima, are found more slowly.

Using the acquisition function, the parameter optimiser determines updated parameter values. These parameters are those for which the acquisition function has a maximum value. Alternatively, a less computationally estimate of this maximum and its parameters associated with them may be used.

The parameters of the measurement processes are set to these updated values. One or more further results are then collected using the measurement processes.

Optionally, these further results, in combination with the previously obtained results, may be used to update the parameters again using the acquisition function. These steps of receiving further results and updating the parameters may be repeated for any number of iterations. For instance, they may be repeated for a number of iterations set by the user, until a given amount of time has passed and/or until a convergence criterion has been reached.

The parameter optimiser receives the results and any number of further results together with the associated parameters. The parameter optimiser derives an updated predictor based on these. For each set of parameter values, the updated predictor provides an estimate of the objective function.

Using the updated predictor, the parameter optimiser determines enhanced parameter values. The enhanced parameters are those for which the updated predictor's estimate of the objective function is at a minimum. It can be verified that the enhanced parameters measure better than the initial values by comparing the value of the objective function on results collected with the parameters set to the initial values and the value of the objective function on results collected with the enhanced parameters.

System Overview

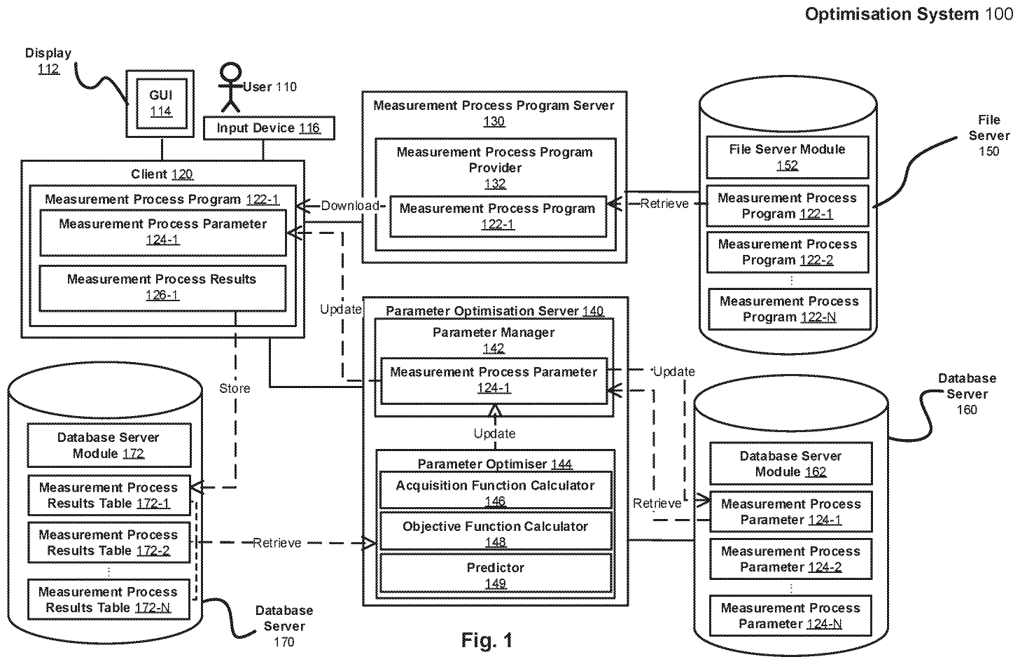

Referring to FIG. 1, an optimisation system 100 for generating enhanced measurement process parameters is shown.

The optimisation system 100 includes a client computing device 120 operable by a human user 110, a measurement process program server 130, a parameter optimisation server 140, a file server 150, a first database server 160 and a second database server 170. The client computing device 120 is configured to communicate with the measurement process program server 130, the parameter optimisation server 140, and the database server 170 through a network. Likewise, the measurement process program server is configured to communicate with the file server 150, and the parameter optimisation server 140 is configured to communicate with the database server over the same or another network. The network(s) may be or include the Internet, an intranet, a local area network, a wireless network, a cellular network and/or a virtual private network. For the sake of clarity, the optimisation system 100 is described as comprising a specific number of computing devices. Any of these may be collocated on a single computing device. For example, two or more of the servers 130, 140, 150, 160, 170 located on a single shared server. Conversely, the servers could be distributed across a number of computing devices.

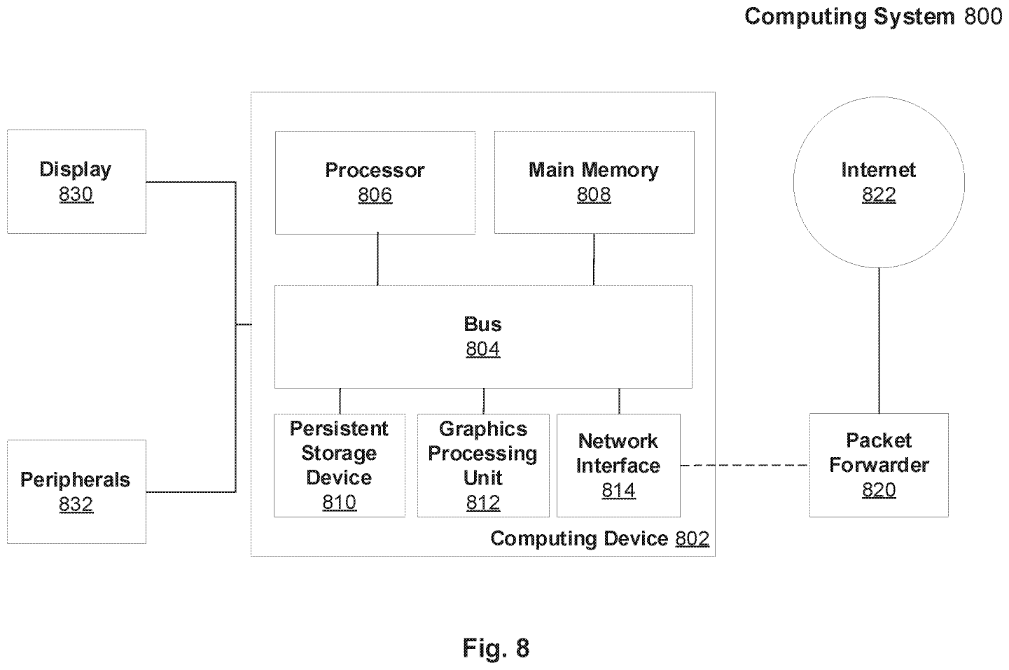

The client computing device 120 can be any suitable computing device for providing the measurement program 122-1 to the user 110. Suitable computing devices include laptop computers, desktop computers, set-top boxes, mobile phones, games consoles, tablet computers, remote desktop client hosts and virtual machine hosts. For instance, the client computing device may include the components of a basic computing system 800 (FIG. 8). The client computing device 120 is connected to a display 112. In some embodiments, the display 112 is integral to the client computing device 120, e.g. a mobile phone or laptop screen, while in others it is peripheral, e.g. a monitor or television.

The client computing device 120 is also connected to an input device 116 which may again be integral or peripheral. The input device 116 may be an input device included in or typically used with generic computing devices such as a keyboard, mouse, touch screen or camera. The input device 116 may also be an input device configured to measure mechanical and/or electrical activity of the human body. Examples include: eye tracking devices; electroencephalogram (EEG) devices, which record electrical activity in the brain using electrodes; and electromagnetic motion tracking devices. While a single input device 116 is shown, most embodiments include multiple input devices, e.g. the client computing device 120 could be connected to a keyboard, a mouse and an eye tracker. While only a single user 110 and client 120 are shown, there may be more than one user and/or more than one client.

Each of the servers 130, 140, 150, 160, 170 include one or more processors (not shown), a memory (not shown) and a network interface (not shown). For example, each of the servers could include some or all of the components of basic computing system 800 (FIG. 8). The one or more processors execute suitable instructions stored in a computer-readable medium, such as the memory. The network interface of each server is used to communicate with the other components of the optimisation system 100 that they are respectively connected to.

The client computing device provides a measurement process program 122-1, configurable by one or more measurement process parameters 124-1, to the user 110. The measurement process program 122-1 presents a suitable graphical user interface (GUI) 114 to the user 110. The user 110 interacts with the measurement process program 122-1 using the input device 116. These interactions are measured by the measurement process program 122-1 which derives measurement process results 126-1 from them. The measurement process program 122-1 stores these results in a measurement process results table 172-1.

Examples of neurocognitive function measurement process programs 122-1 are:
Sustained Attention Measurement Process Programs:
   Task: Visual stimuli, such as pictures or patterns, are presented to the user 110 in turn. The user is asked to provide an input when they detect a specific sub-sequence of visual stimuli. For example, the user could be asked to click when they see a triangle followed by a square and then a circle.
   Results:
      The number of sequences the user successfully recognises.
      The number of times the user indicates a sequence has been seen but it has not been shown.
      How long the user takes to respond, when successful, once the given sequence is shown.
   Relevant neurocognitive functions:
      Working memory: the user must remember the visual stimuli previously presented to them.
      Attention: the user must continuously pay attention to the visual stimuli as they will miss sequences if they do not.
      Visual processing: can the user recognise each visual stimuli in the short time it is presented for.
      Motor control: if the user can respond more quickly after seeing a sequence of stimuli their reaction time may be quicker
   Parameters:
      The length of the sequence: Longer sequences require the user to remember more previous items. Increasing this parameter could increase the demands of the task on working memory.
      The time that each stimulus is shown for: Showing a stimulus for a shorter period requires the user to visually process each stimulus more quickly and reduces the window that they may lose attention for.
      Frequency of target sub-sequences. Lower frequency could require greater ability to sustain attention in the absence of relevant inputs and overt responses.
Block Measurement Process Programs:
   Task: A square area is split in to a number of equally sized smaller squares, e.g. if the length and width of each smaller square is a quarter of that of the larger square then it will be split into 16 smaller squares. A number of blocks consisting of one or more of the square areas are presented to the user 110. The user can remove blocks by clicking on them. The blocks also act under ‘gravity’ i.e. if the block(s) underneath a block are removed then the block will fall. The user is requested to put the blocks in to a given configuration within a limited number of clicks. The user repeats this task several times for different initial and desired block configurations.
   Results:
      The number of tasks the user successfully completes.
      The time the user takes to complete each task.
   Relevant neurocognitive functions:
      Spatial visualisation: the user must visualise what the square area will look like after a block is removed.
      Planning: the user must decide on a limited number of steps to achieve the desired configuration.
   Parameters:
      The number of blocks: displaying more blocks requires the user to visualise a more complex situation.
      The number of clicks: if more clicks are needed to reach the desired configuration the user must make more accurate selections.
      The expected number of falls: falls, and their consequences, are more difficult to predict and visualise than simple removals. These require planning, i.e., the internal mental simulation of multiple spatial reconfigurations.
Switching Stroop Measurement Process Programs:
   Task: The task is a more complex version of the Stroop test. The user is presented with a square coloured red or blue, text stating ‘colour’ or ‘text’, and two boxes. One box contains the word ‘RED’ and another contains the word ‘BLUE’. One of the words is coloured blue and the other is coloured red. If the text states ‘colour’, the user is tasked with clicking on the word coloured the same as the square, e.g. clicking on the word coloured red if the square is red. If the text states ‘text’, the user is tasked with clicking on the word that corresponds to the square's colour, e.g. clicking on the word ‘BLUE’ if the square is blue. The user performs a number of these tasks. In these tasks, any of the square's colour, the colouring of each respective word, the position of each word, or whether ‘colour’ or ‘text’ is displayed may change.
   Results:
      The number of tasks the user is successful at.
      The time the user takes to complete each task.
   Relevant neurocognitive functions:
      Inhibition: the user has to pay attention to one feature of each word, its text or its colour, while being able to filter out or ‘inhibit’ the other.
      Cognitive flexibility: when the criteria changes from ‘text’ to ‘colour’ or vice versa, what information the user has to pay attention to and what information they must filter out changes.
   Parameters:
      The frequency with which the text changes between ‘text’ and ‘colour’: adapting to more frequent changes requires greater cognitive flexibility.
      The frequency with which the colour of the word and the text of the word are incongruent: modulates the balance of inhibition control vs. routine response.

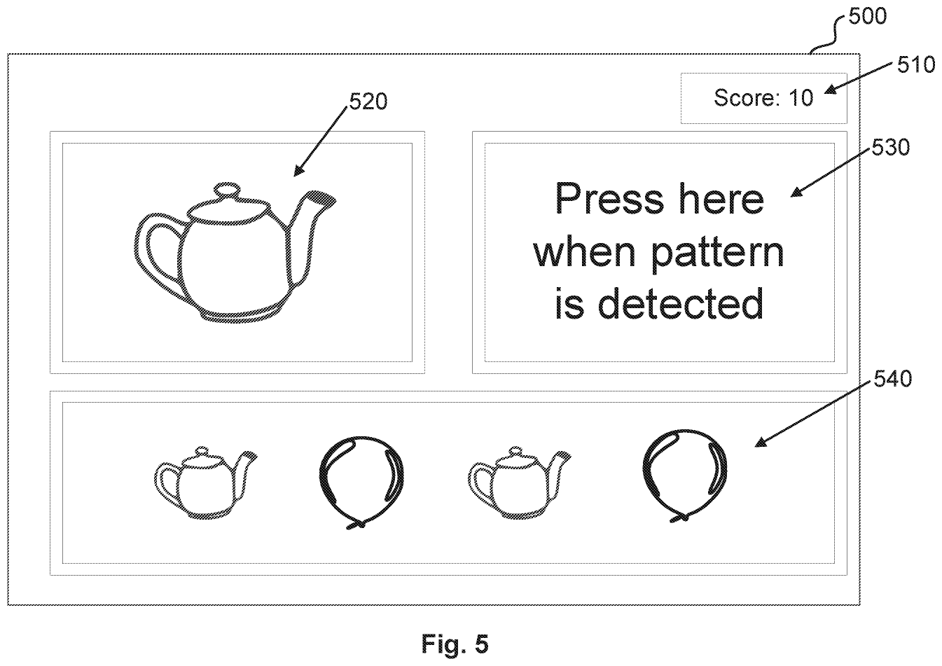

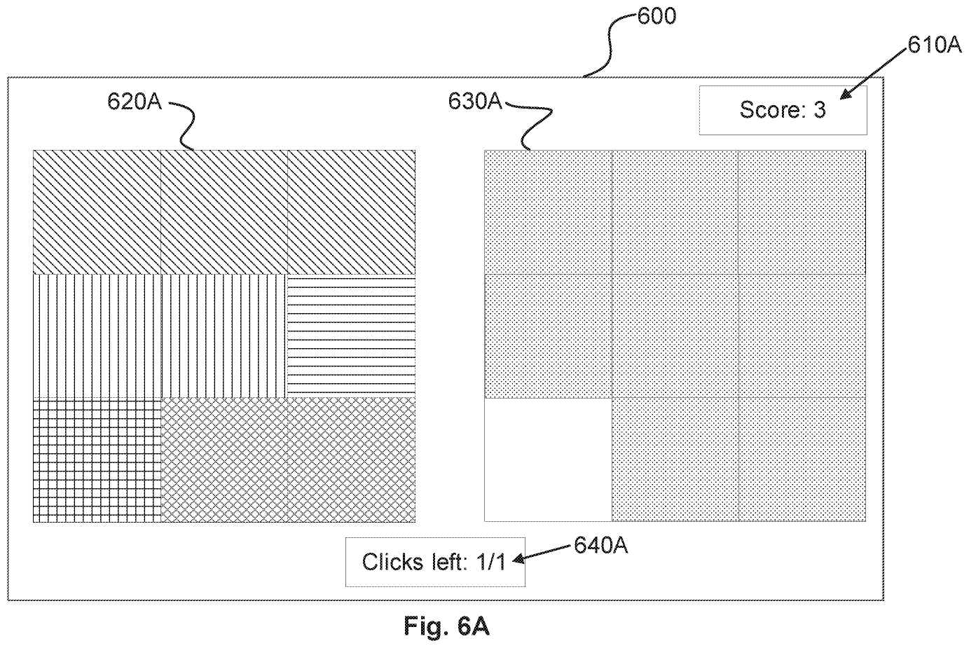

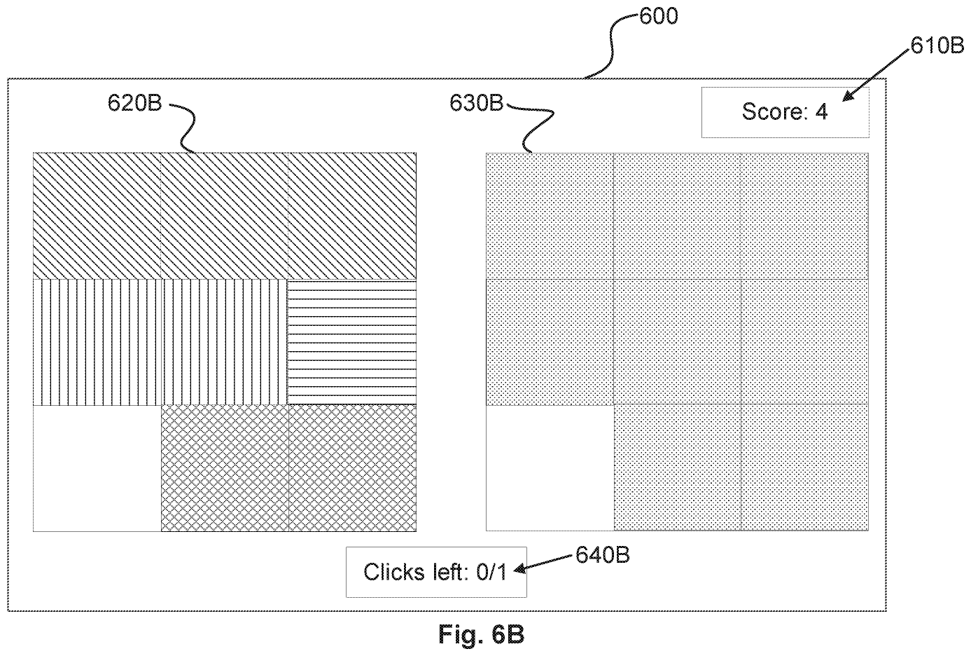

Embodiments of Sustained Attention Measurement Process Programs will be described in more detail with respect to FIG. 5. Embodiments of Block Measurement Process Programs will be described in more details with respect to FIGS. 6A and 6B.

The measurement process program provider 132 is a computer program on the measurement process program server that retrieves measurement process programs 122 from the file server 150 and provides them to the client 120. The retrieved measurement process programs 122-1 may be retrieved as interpretable code, bytecode or native binaries. In some embodiments, the measurement process program provider 132 is responsible for providing each of the measurement process programs 122 to the client in turn. In other embodiments, the client is responsible for doing so and requests each program 122 from the provider 132 in turn.

The measurement process programs provided in turn may be all of the measurement process programs 122 or may be a subset of the measurement process programs 122. The subset provided may be determined using initial measurement process results, wherein these initial results have been collected for all of the measurement process programs 122. The subset selected may be those measurement process programs 122 enabling the derived latent variables to be best discretised, i.e. the subset may be the measurement process programs 122, where the derived measurements of one latent variable have the least impact on the measurements of the other latent variables. The subset selected may also be those measurement process programs 122 whose results are least correlated with those measurement processes to minimise redundancy in the data collected. The selected subset may be derived using a component analysis method, e.g. factor analysis, principle component analysis or independent component analysis.

The parameter manager 142 is a program on the parameter optimisation server 140. It is responsible for retrieving measurement process parameters 124 from the database server 160, and for setting the measurement process parameters 124-1 on the client to these values. The parameter manager also receives updated and enhanced parameters from the parameter optimiser 144. When it receives the updated parameters, it updates the values of the parameters on the client 120 and stores the updated parameters in the database 160. When it receives the enhanced parameters, it stores the enhanced parameters in the database 160 and may update the values of the parameters on the client 120.

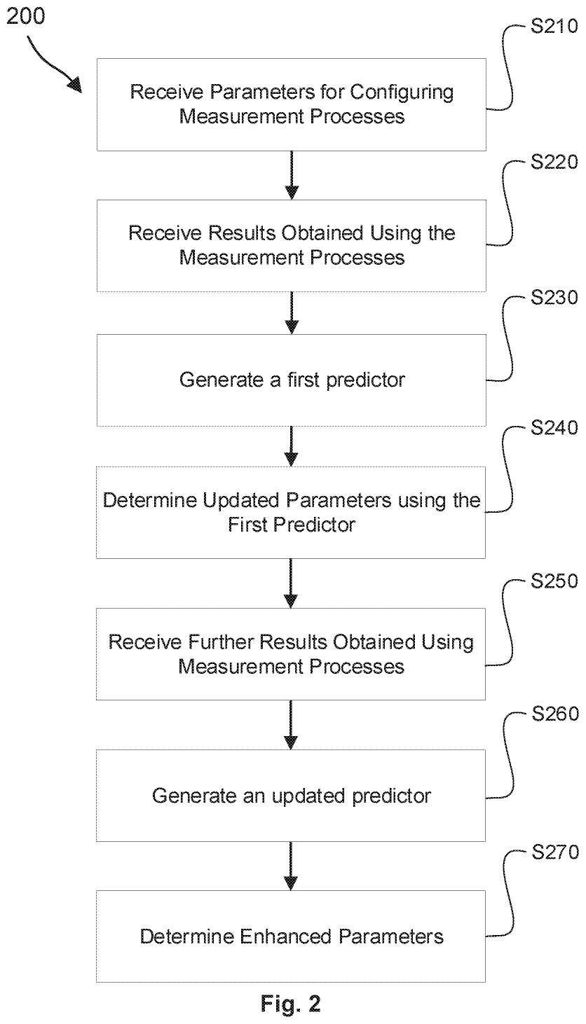

The parameter optimiser 144 is another computer program on the parameter optimisation server 140. It is responsible for generating updated measurement process parameters and enhanced measurement process parameters, and providing them to the parameter manager. The updated measurement process parameters are determined using at least the acquisition function calculator 146, the objective function calculator 148, the predictor 149, and results retrieved from the measurement process results tables 172. The parameters are updated one or more times with the goal of locating improved measurement process parameters 124 for measuring the latent variables. For example, measurement process parameters that can be used to derive more precise values for the latent variables. The parameters 124 are updated, according to an exploration-exploitation trade-off, and the predictor 149 is updated until a stopping criterion is reached. The stopping criterion may be any number of: a limit to the maximum number of updates being reach, a time limit being reached and/or a threshold accuracy being reached. Once the stopping criterion has been reached, enhanced parameters are determined using the predictor 149. These enhanced parameters enable improved measures of the latent variables to be derived from the measurement process results 126. For example, the enhanced measurement process parameters may enable the latent variables to be derived more precisely from the measurement process results 126 than the initial measurement process parameters. Details of how the parameter optimiser 144 may perform these functions are described in relation to method 200 with respect to FIG. 2.

The file server 150 includes a file server module 152 that is able to store and retrieve the measurement process programs 122. The file server module 152 could take any suitable form such as an FTP server module, a HTTP server module, a Server Message Block server, often used in Windows® local area networks and virtual private networks, or a network file system server, often used in local area or virtual private networks of Unix® or Unix-like systems. Alternatively, the file server module 152 may be a database server module that is capable of storing and retrieving the measurement process programs 122. In the case of interpretable code, a database server module that can retrieve and store text data is sufficient. In the case of bytecode and native binaries, a database capable of binary data retrieval and storage is needed.

The database server 160 includes a database server module 162 that is able to store and retrieve the measurement process parameters. The database server module 162 could take any suitable form such as a SQL server module, a NoSQL server module or a flat file database module.

The database server 170 includes a database server module 172 that is able to store, update and retrieve the measurement process result tables. The database server module 172 could take any suitable form such as a SQL server module, a NoSQL server module or a flat file database module.

Separate file and database servers 150, 160, 170 and associated server modules 152, 162, 172 have been described for clarity. However, in some embodiments, a common server and server module may be used to implement the functions of these servers. Likewise, the file and database servers 150, 160, 170 need not be single devices and may be distributed or clustered servers.

Parameter Optimisation Method

FIG. 2 is a flow diagram of an example method by which measurement process parameters are optimised. The method 200 is performed by executing computer-readable instructions using one or more processors of one or more computing devices, e.g., the basic computing device 800 (FIG. 8). In some embodiments, the one or more computing devices are the parameter optimisation server 140. In other embodiments, the one or more computing devices are all or some portion of the devices of the optimisation system 100.

S210 receives parameters for configuring a set of measurement processes. The parameters can be in any format allowing values suitable for configuring the measurement processes to be obtained using them. For example, a defined transformation, such as normalisation or unit conversion, may be required before the parameters are suitable for configuring the measurement processes. Similarly, the received parameters may be in a compressed form so may require decompression before they can be used for measurement processes. The parameters may also need to be extracted from a wider document, e.g. from a markup language file or a spreadsheet.

These parameters are received using any suitable mechanism. The parameters may be actively retrieved by, for example, making a remote procedure call (RPC), calling a Representational State Transfer (REST) service, making a database request or reading them from a file. The parameters may also be passively received. For example, by receiving one or more network packets, such as TCP or UDP packets, message queue events e.g. Advanced Message Queueing Protocol events, or function call parameters.

S220 receives results obtained using the set of measurement processes. These results have been obtained when the set of measurement processes were configured using the parameters. The results can be in any form such that they are able to be used for the subsequent steps of the method 200. For example, unnecessary data or outliers may have been removed from the results, or the results may have been normalised or subject to a mathematical transformation. The results may also be in any suitable data format, e.g. database data, in-memory data structures, markup language or text.

S220 receives these results using any suitable mechanism. In some embodiments, the results are actively retrieved by, for example, making a remote procedure call (RPC), calling a Representational State Transfer (REST) service, making a database request or reading them from a file. In other embodiments, the results are passively received. For example, one or more network packets, such as TCP or UDP packets, message queue events e.g. Advanced Message Queueing Protocol events, or function call parameters are received.

S230 generates a predictor that predicts a value of an objective function for one or more unsampled parameter sets, i.e. parameter sets that the measurement process has not been configured with and that results are not available for. The predictor also provides an uncertainty value, or a confidence measure, for each of these unsampled parameter sets. The predictor is generated using the values of the objective function for the received parameters. These objective function values are calculated using the received one or more results.

As a first step in calculating the objective function values, latent variables may be derived from the results. In some embodiments, each of these latent variables correspond to different aspects of neurocognitive function. The latent variables may be derived by applying a function to the results or by multiplying the results by a matrix. For example, if a data structure storing the results, e.g. an array, is represented using a vector x̅ then a vector y̅, containing the latent variables, may be derived as t(x̅) or as Ax̅, where t is a function transforming the results in to latent variables and A is a suitable transformation matrix. The matrix A may be derived using any suitable method, e.g. factor analysis, principle component analysis or independent component analysis.

An example of a suitable objective function is the squared difference, or squared Euclidean distance, between the observed correlation matrix and a cross correlation matrix for the latent variables that captures the desired structure. An example of desired would be strong clustering within the correlation matrix, such that within cluster correlations approach 1 and between cluster correlations approach 0. Various types of correlation could be used, e.g. Pearson, Spearman or Kendall tau. Alternatives to correlation could also be used, e.g. the mutual information or Kullback-Leibler divergence.

Using mathematical notation, where there are n latent variables, the squared difference between the target and observed correlation matrices may be represented as:

$${\sum }_{i = 1}^{n} {\sum }_{j = 1}^{n} {( {ρ}_{{y}_{i} {y}_{j}}^{observed} - {ρ}_{{y}_{i} {y}_{j}}^{target} )}^{2}$$

In some embodiments, the target cross correlation matrix is the identity matrix. In other embodiments, the target cross correlation matrix contains known correlations between the one or more latent variables, e.g. known relationships between the various aspects of neurocognitive function. While the squared difference is used by way of example, any other suitable metric could be used, e.g. Manhattan distance or Chebyshev distance. It should also be noted that while the objective function is illustrated as being applied to the derived latent variables, it could also be applied to the results x̅.

Another example of a suitable objective function could be the distance of the observed latent variables from those predicted as a function of the other latent variables using a theoretical model. Such a model could also derive values for the latent variables based on other data, e.g. EEG or fMRI data. For example, a model function t(y̅) could derive a value tᵢ for each yᵢ based on all other elements of y̅, i.e. all elements except yᵢ itself. If the results of t(y̅) are represented as a vector t̅, an example objective function would be:

$${\sum }_{i = 1}^{n} {( {y}_{i} - {t}_{i} )}^{2}$$

The predictor is then generated using the parameters and objective function values by way of a suitable method. In many embodiments, forms of Bayesian inference, e.g. Gaussian process regression, student-t process regression or Bayesian linear regression, are used to produce the predictor. However, alternatives exist. For example, most neural networks cannot provide uncertainty values but a small number of specialised implementations are able to.

Bayesian inference assumes that the true objective function values for the received results, ō, are produced by one of a number of predictor functions, ƒ(ī), where ī are the parameters values. A prior, P(ƒ), over a, potentially infinite, set of functions is chosen. The prior is a probability function that describes our prior beliefs about the predictor function before the data, D={ī, ō}, is taken in to account. For example, we may know that the objective function oscillates, is constrained within a given range or has a given number of inflections. The prior can also be understood as the probability of the predictor function being a given function ƒ. We then use the data, D, to find a posterior probability distribution, P(ƒ|D), over the functions. The posterior describes beliefs about the true objective function after the data has been taken into account. In many cases, the posterior is calculated using Bayes' rule:

$$P ( f \vert D ) = \frac{P ( D \vert f ) P ( f )}{P ( D )} α P ( D \vert f ) P ( f )$$

P(D) does not need to be known as it is a constant value. As we know that the posterior across all functions sums (or, for an infinite number of functions, integrates) to one, this constant can be derived as:

$$P ( D ) = \frac{1}{{Σ}_{f} P ( D \vert f ) P ( f )}$$

From the posterior, the value of the objective function for any parameter set ī can be predicted as the expectancy over the posterior. This predictor is referred to as the mean function, μ(ī), as it provides a mean prediction of the objective function. The mean function is:

$$μ ( \overline{i} ) = {\sum }_{f} f ( \overline{i} ) P ( f \vert D )$$

The posterior is usable as a measure of the uncertainty of our prediction of the value of the objective function for ī. Other measures of uncertainty can also be derived from the posterior. For example, the standard deviation:

$$σ ( \overline{i} ) = \sqrt{{\sum }_{f} P ( f \vert D ) {( f ( \overline{i} ) - μ ( \overline{i} ) )}^{2}}$$

At least by providing the mean predictions and associated uncertainties, Bayesian inference can be used to generate the predictor.

S240 determines updated parameters using the predictor. S240 determines the updated parameters by finding where, or at least a best estimate of where, the value of an acquisition function is at an optimum. The optimum is a maxima or minima depending on whether the acquisition function is defined as a utility function, a function to maximise, or a loss function, a function to minimise. For the sake of clarity, maximising the acquisition function will be referred to hereinafter, but it should be understood that these references are non-limiting.

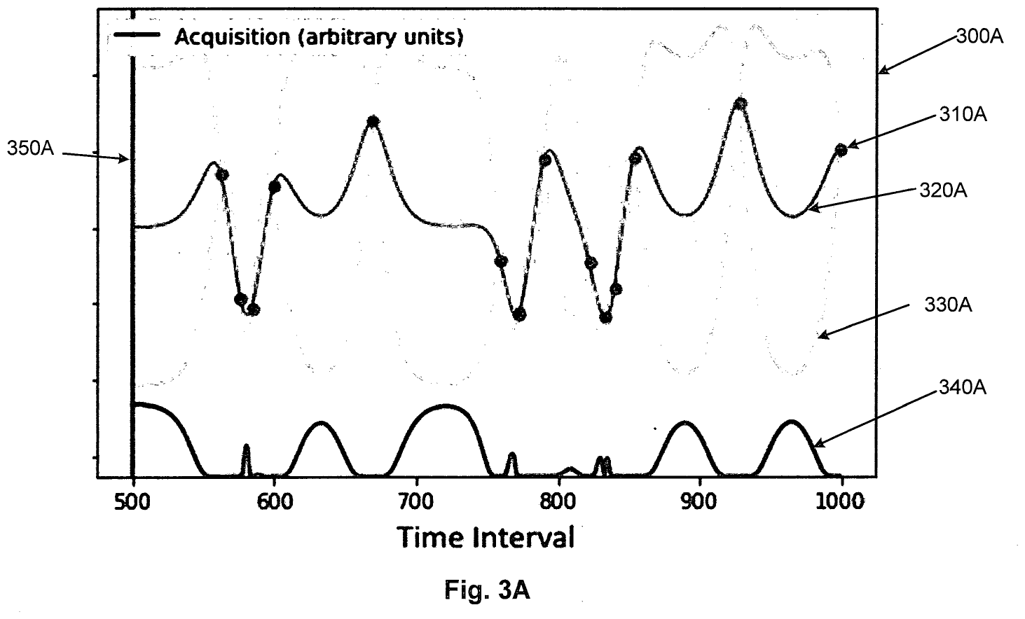

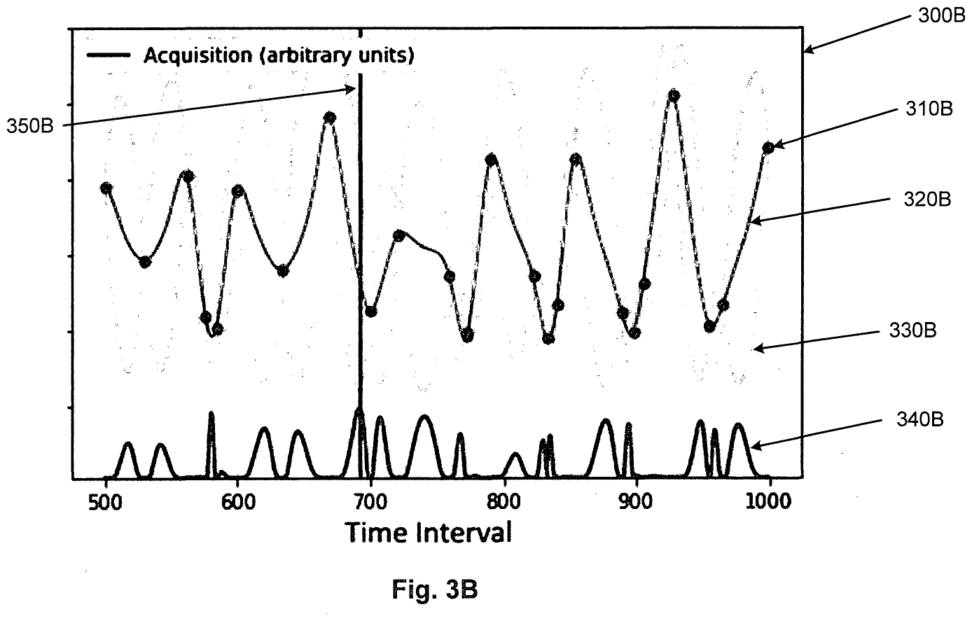

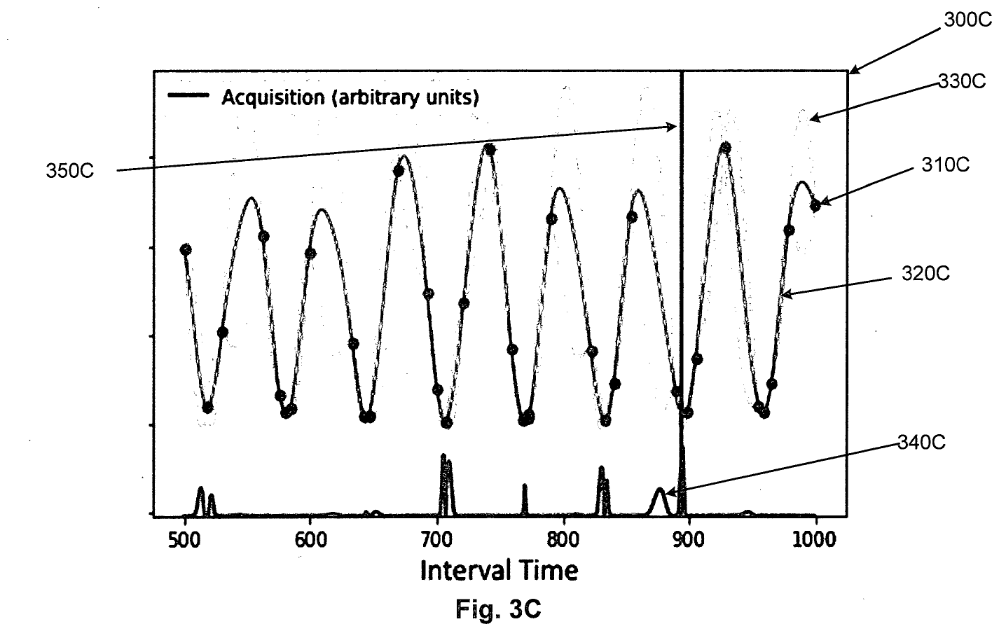

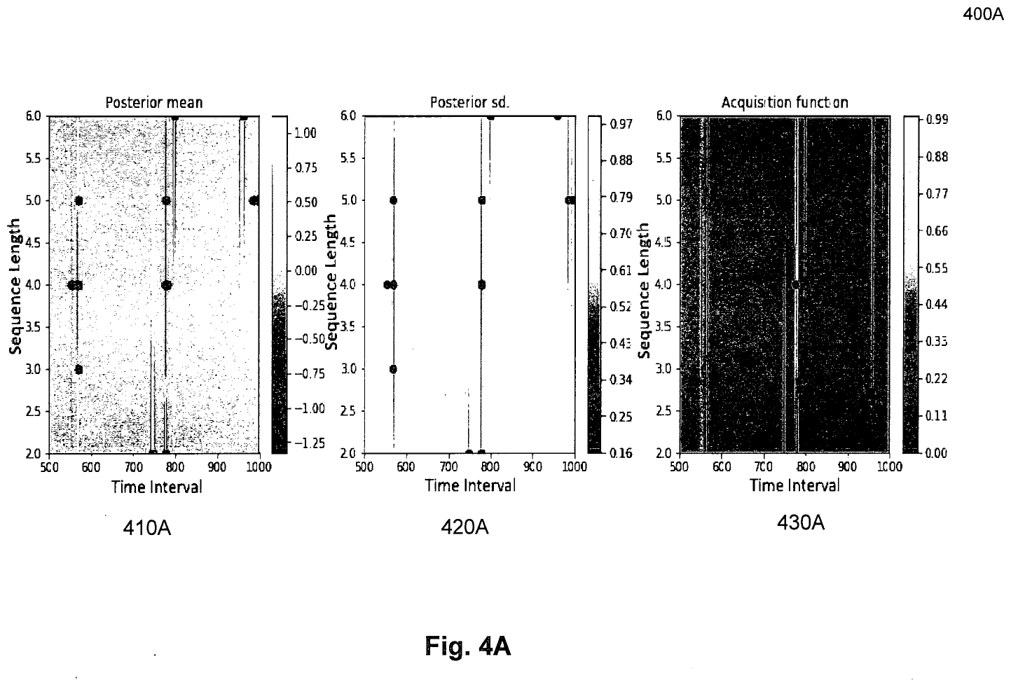

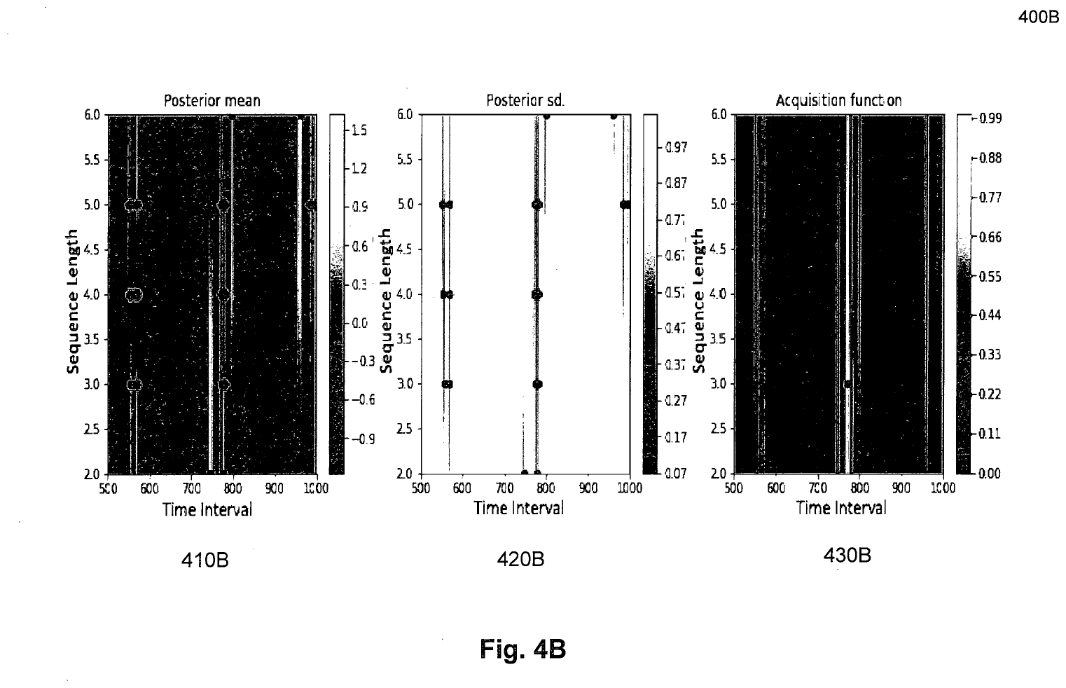

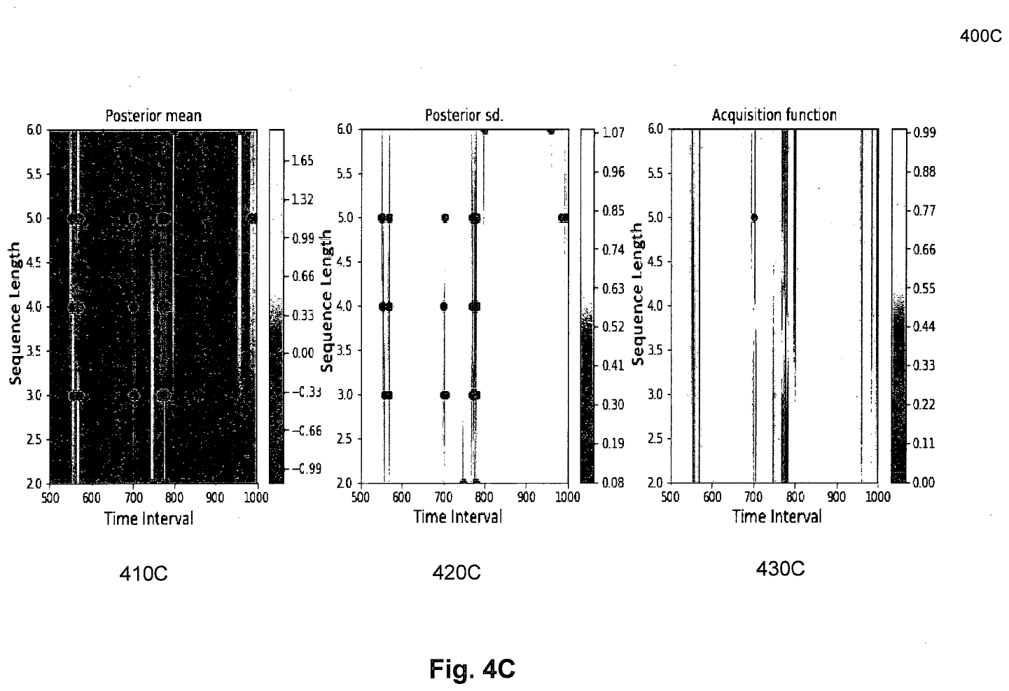

The acquisition function provides a so-called “usefulness” for each of a plurality of different parameter sets. So, the acquisition function can be used to locate the most useful parameters i.e. those with the greatest usefulness. This usefulness is derived based on the predictor. This term will be explained further with respect to FIG. 3 and FIG. 4.

In Bayesian inference embodiments, the acquisition function uses the posterior distribution, P(ƒ|D), to derive these usefulness values.

One example is the Expected Improvement acquisition function. The Expected Improvement acquisition function determines which parameter sets are expected, according to the posterior distribution, to lead to the greatest amount of improvement.

Where ī_{best} is the set of parameter values for which the mean is predicted to be lowest, expected improvement, EI, is calculated as:

EI(ī)=E_{P(ƒ|D)}[max(0,μ(ī_{best})−ƒ(ī)]

The updated parameters would, therefore, be:

$${\overline{i}}_{u p d a t e d} = {arg min}_{\overline{i}} EI ( \overline{i} )$$

An alternative acquisition function is the probability of improvement. The probability of improvement is the probability that a given parameter set ī results in a lower objective function value according to the posterior distribution:

PI(ī)=P_{P(ƒ|D)}(ƒ(ī)<μ(ī_{best}))

In some embodiments, the acquisition function is parametrised such that a desired exploration-exploitation trade-off, as previously described, can be chosen. For example, the acquisition function may be a parametrised variant of the expected improvement or probability of improvement functions.

S250 receives one or more further results obtained using the set of measurement processes. These further results have been obtained when the set of measurement process was configured using the updated parameters. As for the results, the further results can be in any form such that they are able to be used for the subsequent steps of the method 200. Several examples are described with respect to S220. As before, these further results are received using any suitable mechanism. Several examples are described with respect to S220.

S260 generates an updated predictor. Values of the objective function for the updated parameters are used to generate the updated predictor. The received one or more further results are used to calculate these objective function values. The updated predictor may take any of the forms described with respect to the predictor in S230. The updated predictor may also be derived by any of these same methods, e.g. Bayesian inference. The updated predictor may be generated by updating the predictor, or may be a newly generated predictor.

In the case of Bayesian inference, there are at least two methods usable for generating the posterior distribution for the updated predictor.

The first method is generating the posterior distribution for the updated predictor as for S230 using the prior and a dataset but, in this case, the dataset would contain the calculated objective function values for both the received parameters and the updated parameters. Where Dᵣ comprises the received parameters and associated objective function values, {īᵣ, ōₜ}, and Dᵤ comprises the updated parameters and associated objective function values, {īᵤ, ōᵤ}, the posterior may be calculated as:

$$P ( f \vert {D}_{u} , {D}_{r} ) = \frac{P ( {D}_{u} , {D}_{r} \vert f ) P ( f )}{P ( {D}_{u} , {D}_{r} )} α P ( {D}_{u} , {D}_{r} \vert f ) P ( f )$$

The second method is generating the posterior distribution of the updated predictor by using the posterior of the predictor, P(ƒ|Dᵣ), as the prior, i.e.:

$$P ( f \vert {D}_{u} , {D}_{r} ) = \frac{P ( {D}_{u} \vert f ) P ( f \vert {D}_{r} )}{P ( {D}_{u} )} α P ( {D}_{u} \vert f ) P ( f \vert {D}_{r} )$$

This second method may use less computational resources than the first as it reuses the posterior distribution already calculated for the predictor.

In some instances, the choice between the first and second method is made dynamically depending on the situation. For example, suppose the posterior distribution generated by the predictor is cached in memory for a limited period of time before being deleted. If the posterior distribution generated by the predictor is still in memory, the second method is used to generate the updated predictor. Otherwise, the second method is used.

S270 determines enhanced parameters using the updated predictor. The enhanced parameters are those that the updated predictor predicts to have the lowest objective function values, i.e. the minima of the predictor's estimate of the objective function's value. In the case of the Bayesian inference models described, the enhanced parameters, are typically those where the mean prediction of the objective function is lowest:

$${\overline{i}}_{e n h a n c e d} = {arg min}_{\overline{i}} μ ( \overline{i} )$$

In a subsequent step, the improvement provided by the enhanced parameter values can be verified. Additional measurement process results obtained using the enhanced parameter values can be received and an objective function value calculated for them. If this value is lower than the objective function value for the measurement process when using the initial parameters, the measurement processes have been successfully optimised.

The steps 240 to 260, or 240 to 270, may be repeated for several iterations, with the updated predictor of one iteration used as the predictor of the next iteration. For instance, they may be repeated for a number of iterations set by the user, until a given amount of time has passed and/or until a convergence criterion has been reached. Examples of convergence criteria include: the same enhanced parameter set being determined for multiple iterations; the difference between the determined enhanced parameters being below a threshold for multiple iterations; and the prediction of the best objective function value being below some desired value.

Examples of Bayesian Inference Methods

FIGS. 3 and 4 relate to embodiments where a particular form of Bayesian inference, Gaussian process regression, is used to implement the system and methods herein described. For example, it is a technique for generating a predictor and updating the predictor and/or generating an updated predictor, e.g. predictor 149. These embodiments may use a Gaussian Process software library such as GPy, GPFlow, scikit-learn, or libGP to implement Gaussian Process regression.

Gaussian processes are collections of random variables. Any finite number of these random variables have a joint Gaussian distribution. Gaussian processes are useful for Bayesian inference as they can describe a distribution over functions, e.g. prior P(ƒ) and posterior P(ƒ|D).

As any finite number of random variables of a Gaussian process have a joint Gaussian distribution, it can be entirely specified by its mean and covariance functions. The mean function of a Gaussian process of a distribution over objective functions ƒ accepting a parameter set ī is defined as,

μ(ī)=E[ƒ(ī)]

the covariance function of this Gaussian process is defined as,

k(ī,i̅′̅)=E[(ƒ(ī)−μ(ī))(ƒ(i̅′̅)−μ(i̅′̅),

where i̅′̅ is a second parameter set, and the Gaussian process is written as

ƒ(ī)˜GP(μ(ī),k(ī,i̅′̅)).

In many embodiments, the mean function of the Gaussian process, μ(ī), is set to 0 for several reasons. First, it is a sensible prior, i.e. before data has been considered, it is logical to assume that the mean across the function distribution is 0. Data is also often normalized to ensure this assumption is sensible. Second, if 0 is used as a mean, fewer computational resources are needed to perform Gaussian process regression. The following description assumes a mean function of 0 for ease of explanation. However, another mean function can be used.

The covariance function, k(ī, i̅′̅), of a Gaussian Process is known as a kernel. There are a wide number of kernels which may be used for the Gaussian Process. Examples of kernel functions include: squared exponential kernels (also referred to as radial basis function kernels), rational quadratic kernels, periodic kernels and squared log kernels. To illustrate, an example of a squared exponential kernel is:

k(ī,i̅′̅)=exp(−½(1̅−i̅′̅)²)

In addition, several Gaussian process libraries provide functionality for automatically choosing a suitable kernel function according to a chosen trade-off between model parsimony and the fit of the model to the observed data. The kernel may also be chosen based on the exploitation-exploration trade-off desired as the kernel affects estimations of uncertainty.

Using the Gaussian Process model described, the joint distribution of objective function values, ƒ̅, for sampled parameters I, with a set of objective function value predictions, ƒ̅_̅{̅*̅}̅ for a matrix of unsampled parameters I_{*} is:

$$[ \frac{\overline{f}}{{f}_{*}} ] ∼ N ( 0 , [ \begin{matrix}K ( I , I ) & K ( I , {I}_{*} ) \\ K ( {I}_{*} , I ) & K ( {I}_{*} , {I}_{*} )\end{matrix} ]$$

The parameter matrices, I and I_{*}, comprise row vectors of parameters. Each row vector has the parameters for which the objective function value in the corresponding row of ƒ̅ was obtained. The matrix notation above, K(X, Y), denotes a matrix containing the covariance for all pairs of the parameter sets contained in matrices X and Y, i.e. K(I,I_{*}) is the covariance evaluated at all pairs of sampled parameter sets with unsampled parameter sets.

Given the distribution above, it is possible to use linear algebra to derive a posterior distribution for ƒ̅_̅{̅*̅}̅, the predictions of function values for unsampled parameter sets. In mathematical terms, the posterior distribution for ƒ̅_̅{̅*̅}̅ is:

ƒ̅_̅{̅*̅}̅|I_{*},Iƒ̅˜N(K(I_{*},I)K(I,I)⁻¹ƒ̅,K(I_{*},I_{*})−K(I_{*},I)K(I,I)⁻¹K(I,I_{*}))

This method of deriving a posterior distribution may be used for the first and/or updated predictors of the method herein described, or for deriving and updating the predictor 149. It may also be used to implement any of the steps of determining predictors for any system or method.

It should be noted that the above description of Gaussian process regression is a simple example provided for the purposes of explanation. Any suitable variation of Gaussian process regression could be used.

Referring to FIG. 3A to 3C, plots 300 are shown which illustrate several iterations of a Bayesian optimisation method, such as the method 200 described with reference to FIG. 2, using Gaussian progress regression, applied to optimising the time interval parameter of a sustained attention measurement process program. The plots 300 are exemplary and, as such, the ‘sampled’ data illustrated in the plots 300 have been generated using an arbitrary function, i.e. it is not experimental data. The x-axis is the time interval in milliseconds and the y-axis is the predicted, or sampled, value of the objective function.

In the plots 300, round circular points, such as point 310, represent sampled points of the objective function. These correspond to the results and further results of the method 200.

The function lines 320 are mean estimates of the objective function value for each interval time in the range 500 ms-1000 ms derived based on the sampled points using Gaussian process regression. The function line equates to the estimates of the objective function value for unsampled parameters provided by the predictor of method 200, e.g. predictor 149.

The shaded areas 330 represent the uncertainty in the mean estimates of the objective function values. Specifically, the shaded areas 330 show the range two standard deviations from the mean estimate for the predicted function value for each time interval. As is visible in the plots 300, the standard deviation in the predicted objective function value is comparatively low for values at or near to sampled time values but is comparatively high for those far from any sampled values.

The lines 340 show the value of the acquisition function, in this case expected improvement, for interval times in the range 500-1000 ms. It is clear from the plots 300 that the acquisition function is dependent on both the uncertainty in its predictions, e.g. their standard deviation, and the current mean estimate of the objective function for any given interval time.

The vertical lines 350 represent the interval time for which the acquisition function is at a maximum in each respective iteration. These indicate the next interval time, i.e. updated parameter(s), to be sampled.

Plot 300A illustrates the estimated means and standard deviations after a small number of points have been sampled. In this plot 300A, it is clear that the uncertainty 330A for a large number of interval times is substantial, and that the prediction of the true objective function provided by the mean estimate, 320A, is far from the true objective function.

In subsequent iterations, represented by plots 300B and 300C, more points are sampled according to the acquisition function. In each of these iterations the prediction of the true objective function, represented by lines 320B and 320C, improves drastically, and the standard deviation, i.e. the uncertainty, also substantially decreases. The uncertainty reduces most for those points having low mean estimates.

Plots 400 relating to FIGS. 4A-4C provide an illustration in a two-dimensional case. In this example, both the time interval between each stimulus in the sustained attention task and the sequence length used are being optimised. The plots 400 are exemplary and, as such, the ‘sampled’ data illustrated in the plots 400 have been generated using an arbitrary function, i.e. it is not experimental data.

Mean subplots 410 are plots illustrating the standard deviation of the estimate of the objective function for each combination of sequence length and time interval. Their x-axis are the time interval, their y-axis are the sequence length and their estimate of the mean is represented using a heat map. A key for the heat map is shown on the right-hand side of the subplots 410. The sampled points are shown in these subplots as black dots.

Standard deviation subplots 420 are plots illustrating the standard deviation of the estimate of the objective function for each combination of sequence length and time interval. Their x-axis are the time interval, their y-axis are the sequence length and their estimate of the standard deviation is represented using a heat map. A key for the heat map is shown on the right-hand side of the subplots 420. The sampled points are shown in these subplots as black dots.

Acquisition function subplots 430 are plots illustrating the value of the acquisition function for each combination of sequence length and time interval. Their x-axis are the time interval, their y-axis are the sequence length and their values for the acquisition function are represented using a heat map. A key for the heatmap is shown on the right-hand side of the subplots 430.

Plot 400A illustrates the estimated means and standard deviations after a small number of points have been sampled. In this plot 400A, it is clear that the standard deviations 420A for a large number of interval times and sequence length combinations are substantial, and that the prediction of the true objective function provided by the mean estimate, 410A, is far from the true objective function.

In subsequent iterations, illustrated in plots 400B and 400C, more points are sampled according to the acquisition function. In each of these iterations, the prediction of the true objective function, represented by subplots 420B and 400C, improves and the standard deviation, i.e. the uncertainty, also decreases. Unlike, the one-dimensional example, even in the final iteration 400C the standard deviation for a large percentage of the sample space is still substantial. However, those points most useful for locating the true optima, according to the acquisition function, have been sampled. Therefore, the parameters for which its estimate of the mean, i.e. its predictions of the objective function value, is lowest, corresponding to the enhanced parameters of method 200, are close to the true optimum parameters in this iteration. This demonstrates the power of Bayesian inference methods in locating optimum values using a small amount of data.

Sustained Attention Measurement Process Program

Referring to FIG. 5, an illustration of a graphical user interface 500 for the Sustained Attention Measurement Process Program is shown.

As hereinbefore described, in this measurement process program, visual stimuli 520 are presented to a user in turn. The user is asked to provide an input, e.g. clicking or tapping in a box 530, upon seeing a given sequence 540 of visual stimuli. Upon successfully doing so, the user's score 510 is incremented. In some embodiments, the user's score is decremented if they click when the pattern has not been shown.

The graphical user interface 500 is implementable using any suitable technology. It may be implemented as any of: a web application interface, a native desktop application interface, a native mobile phone application interface, or an interface produced on an LCD screen by electrical signals from a microcontroller.

The score 510 provides feedback to the user on their performance. The primary purpose of the score box is to incentivise the user. In some embodiments, the score 510 corresponds to the results collected by the measurement process program. Higher scores may also correspond to better neurocognitive function. In other embodiments, the scores do not correspond to the results collected but are some simpler measure sufficient to motivate the user, e.g. a count of successful and failed pattern recognitions. This simpler measure does not account for reaction time, which is collected by the measurement process program.

Visual stimuli 520 are displayed to the user sequentially. The time each visual stimuli 520 is displayed for is a configurable parameter of the measurement process program. In the embodiment illustrated, the visual stimulus 520 being displayed is a picture of a teapot, and all of the visual stimuli 520 displayed are pictures of easily recognisable objects. One reason for this choice is that, for most users, recognising objects is intuitive, unlike recognising abstract patterns. So, using pictures of objects minimises the impact of aspects of neurocognitive function other than attention, e.g. visual processing.

Box 530 contains text instructing the user to click or tap in the box 530 when they recognise the sequential pattern of visual stimuli. In some embodiments, the box 530 does not contain these instructions, and they are presented to the user in another location of the interface 500 or before the user commences the measurement process. In some embodiments, there is not a box 530, and instead the user is asked to press a keyboard or gamepad button on recognising the pattern.

A sequence of visual stimuli 540 that the user must recognise is displayed. In the example illustrated, the sequence 540 is: teapot, balloon, teapot, balloon. The length of this sequence is a configurable parameter.

Block Measurement Process Program

Referring to FIGS. 6A and 6B, an illustration of a graphical user interface 600 for the Block Measurement Process Program is shown.

As previously described, the block measurement process program displays a square area 620 tiled by a number of blocks. Any of the blocks may be removed by clicking on them. The user is tasked with removing blocks until the pattern of tiles matches that shown in a second square area 630. The user has a limited number of clicks 640, i.e. block removals, in which to complete this task. Graphical user interface 600A shows the interface before the user begins the task. Graphical user interface 600B shows the interface after the user successfully completes the task.

The graphical user interface 600 is implementable using any suitable technology. It may be implemented as any of: a web application interface, a native desktop application interface, a native mobile phone application interface, or an interface produced on an LCD screen by electrical signals from a microcontroller.

The score 610 provides feedback to the user on their performance. The primary purpose of the score box is to incentivise the user. In some embodiments, the score 610 corresponds to the results collected by the measurement process program. Higher scores may also correspond to better neurocognitive function. In other embodiments, the scores do not correspond to the results collected but are some simpler measure sufficient to motivate the user, e.g. a count of successfully completed tasks. This simpler measure does not account for task completion time, which is collected by the measurement process program. In this illustration, score 610B has been incremented from that of score 610A as the user has successfully completed the task.

The square area 620 is tiled by a number of blocks. Each block is indicated using hatching, i.e. adjacent squares with the same hatching represent a single block. The blocks in the square area 620 may be removed. In square area 620B, a block has been removed as it has been clicked by the user. In this embodiment, blocks are removed by clicking on them. In other embodiments, other suitable input mechanisms are used to remove the blocks, e.g. touch screen taps, gamepad button presses or keyboard button presses.

The square area 630 shows the tiled pattern that the user is tasked with matching. In this illustration, the user is tasked with removing blocks until there are blocks filling those tiles of square area 620 that correspond to those of square area 630 filled with a dotted square. In this example, the user would complete this task by removing the bottom left block.

The clicks indicator 640 shows the number of clicks which the user has to complete the task in. In some scenarios, such as that shown, this is the minimum number of clicks in which the user is able to complete the task. In others, the user may be permitted to use more clicks than they need but be penalised for every click used over the number required. When starting the task, the click indicator 640A shows that the user has one click remaining of one total allowed clicks. On completing the task, the click indicator 640B shows that the user has no clicks remaining.

Left Cerebral Hemisphere

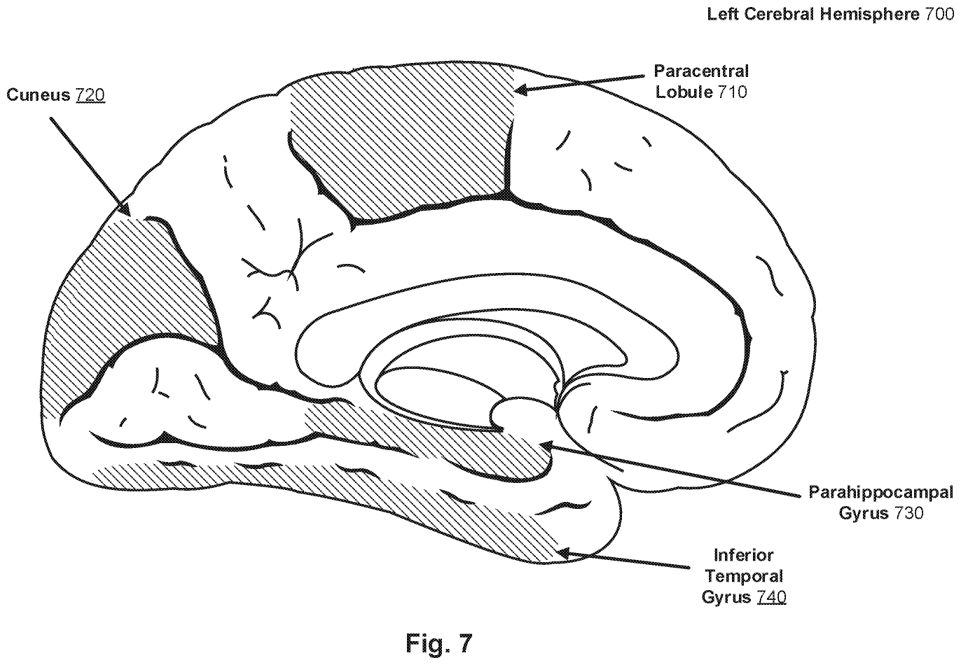

Referring to FIG. 7, an illustration of the left cerebral hemisphere 700 is shown.

The left cerebral hemisphere 700 is one of two hemispheres forming the human cerebrum. The cerebrum is the principal part of the brain in humans, and other vertebrates. It comprises the cerebral cortex and various subcortical structures. The cerebrum is responsible for a range of functions including: cognition, awareness, consciousness and voluntary actions.

Different regions of the left cerebral hemisphere 700 are responsible for different neurocognitive functions. By measuring aspects of neurocognitive function using measurement processes optimised with the systems and methods herein described, e.g. optimisation system 100 and/or optimisation method 120, levels of a given neurocognitive function indicative of injury and/or pathology of the left cerebral hemisphere, or the brain in general, can be detected, e.g. poor working memory. Known associations between the given neurocognitive function and regions of the left cerebral hemisphere, or the brain in general, may be used to infer those regions that are likely to be injured and/or malfunctioning.

By inferring the regions likely to be injured and/or malfunctioning using the optimised neurocognitive function measurement processes, the use of more hazardous and/or expensive procedures for locating injured or malfunctioning regions of the brain can be avoided. Examples of such procedures include functional magnetic resonance imaging (fMRI), computerised tomography (CT) scanning, and positron emission tomography (PET) scanning. Alternatively or in addition, by using the optimised neurocognitive measurement processes to infer those regions of the brain most likely to be injured or malfunctioning, the above procedures may be better targeted by a user of, or software controlling, a respective scanning or imaging device.

The inferred regions may also be used to improve analysis of imagery produced by these procedures. Many procedures produce a large number of detailed images, e.g. brain CT scans produce a large set of images with each image representing a slice of the brain. Therefore, it is difficult for a human radiographer and/or a computational radiography system to know where to focus their analysis. The inferred regions derived from the optimised neurocognitive function measurement process enable the human radiographer and/or the computational radiography system to focus their analysis on those regions that are most likely to be injured and/or malfunctioning. In the case of a computational radiography system, this may lead to significantly less computational resources being used.

To illustrate the relationship between brain regions and neurocognitive function, several regions of the left cerebral hemisphere 700 and the neurocognitive functions to which they relate are described below.

The paracentral lobule 710 is a region of the brain on the medial surface of each cerebral hemisphere 700. The paracentral lobule 710 is a U-Shaped convolution and loops underneath the central sulcus. The paracentral lobule 710 has been found to be associated with the capacity for sustained attention. Therefore, a value significantly below average for a sustained attention latent variable, derived from results of the optimised measurement processes, may be indicative of damage to and/or malfunctioning of the paracentral lobule. In this case, the optimised measurement processes may include a sustained attention measurement program such as that described in relation to measurement process program 122-1 and user interface 500.

The cuneus 720 is a wedge-shaped lobule. It is located on the medial surface of the occipital lobe of the brain, and is between parieto-occiptal sulcus and the calcarine sulcus. The cuneus 720 is involved in processing visual information so damage to the cuneus is likely to result in slower, or otherwise impaired visual processing. Therefore, a value significantly below average for a visual processing latent variable, derived from results of the optimised measurement processes, may be indicative of damage to and/or malfunctioning of the cuneus 720.

The parahippocampal gyrus 730 is a portion of the brain positioned inferior to the hippocampus, and is a major component of the medial temporal lobe. It is part of the limbic system. The parahippocampal gyrus is associated with many cognitive processes including spatial processing and episodic memory. To explain its role on various functions, the parahippocampal gyrus is regarded as being part of a network of brain regions for processing contextual associations. Therefore, a value significantly below average for a contextual association latent variable, derived from results of the optimised measurement processes, may be indicative of damage to and/or malfunctioning of the parahippocampal gyrus 730.

The inferior temporal gyrus 740 is on the temporal lobe of each cerebral hemisphere 710. It is below the middle temporal sulcus and stretches to the inferior sulcus. The inferior temporal gyrus 740 performs higher level visual processing, in particular, it is known to be responsible for object recognition. Therefore, a value significantly below average for an object recognition latent variable, derived from results of the optimised measurement processes, may be indicative of damage to and/or malfunctioning of the inferior temporal gyrus 740.

For ease of illustration, the uses of the systems and methods hereinbefore described are described in relation to regions of the brain. However, the same or similar methods are equally applicable to known associations between neurocognitive function and functional networks spanning several brain regions. Similarly, they are applicable to known associations between neurocognitive function and neurotransmitters.

Computer System

Referring to FIG. 8, it is a schematic diagram illustrating a basic computer system 800 suitable for performing methods herein described, e.g. example the method 200. The basic computing system 800 is also suitable for use as a component of systems herein described, e.g. optimisation system 100.

The components and connections illustrated in computer system 800 are exemplary. In some embodiments, computing systems having different components and/or connections than those of basic computer system 800 are used.

Computing system 800 includes a computing device 802. The computing device is any computing device suitable for implementing the present invention. For example, it may be any of a desktop computer, a laptop computer, a mobile phone or a tablet computer.

Computing device 802 has a bus 804. The bus 804 provides a communication system between the various components of the computing system 802, and directly or indirectly with external components and devices. The bus may be a serial bus or a parallel bus. In many embodiments, the bus 802 includes a plurality of ‘sub-buses’, each of which may itself be serial or parallel. In these embodiments, communication between the plurality of ‘sub-buses’ is mediated by a bus controller.

The computing device 802 also contains one or more processors 806. The processor 806 is coupled to the bus 804. The processor 806 is any device suitable for processing information transferred to the processor 806 via the bus 804. For example, it may be any of a general-purpose microprocessor, a system on a chip (SoC) processor, a Field Programmable Gate Array (FPGA) or an Application Specific Integrated Circuit (ASIC).

Main memory 808, such as a random-access memory (RAM) or other dynamic storage device, is connected to the bus 802. The main memory 808 stores information to be used by and/or provided by the processor 806. It also stores instructions to be executed by the processor 806.

Persistent storage device 810, such as a hard disk drive or solid-state drive, is connected to the bus 804 and persistently stores information. In operation, the processor 806 may retrieve data from the persistent storage device 810 and store it in memory 808. Instructions for execution by the processor 806 may also be loaded in to main memory 808 from persistent storage device 810. Results produced by the processor 806, e.g. by performing methods herein described, may also be stored on the persistent storage device 810.

Graphics processing unit 812 is connected to the bus 804, e.g. over a PCI Express bus. It is responsible for executing instructions for displaying graphical output. The produced graphical output may be transferred via the bus to display 820, which displays the graphical output. The graphical processing unit 812 may also perform non-graphical data processing operations on data received from the main memory 808 and/or the persistent storage device 810. Performing these data processing operations is referred to as General Purpose Graphical Processing Unit (GPGPU) computing. A Graphical Processing Unit 812 is typically able to perform certain operations, particularly highly data parallel computations, in significantly less time than the processor 806. Many quantitative and machine learning methods, including Gaussian Process regression, contain highly data parallel computations such as matrix multiplication. Implementations of these methods, therefore, benefit from performing some portion of their computations on the Graphical Processing Unit 812.

A network interface 814 is connected to the bus 804. It is responsible for two-way communication over a network via a wired or wireless interface. The network interface 814 sends and receives optical, electromagnetic or electrical signals representing digitally encoded data. For example, the network interface 514 may be any of: a wired network interface card, e.g. an ethernet card; a wireless network interface card, e.g. an 802.11 compatible card; a wired modem, e.g. an ADSL modem; or a cellular network modem, e.g. a Long Term Evolution network modem.

The network interface 814 is connected via a wired or wireless link with a packet forwarder 820. In the case of a wired or wireless local area network interface, the packet forwarder 820 may be a device known as a router. In the case of a cellular network interface, the packet forwarder 820 may be a cellular network base station. The packet forwarder 820 receives packets from and transmits them to other devices connected to the packet forwarder, i.e. in a local network, and/or the internet 832.

A display 830 is connected, directly or indirectly, to the bus 804. The display 830 is any device that can be used by the computing device 802 to present content to the user. In many embodiments, it is a visual display, such as a liquid crystal display (LCD) or an organic light emitting diode (OLED) display. However, in other embodiments, the display is non-visual and presents information to the user via another sense modality, such as sound or touch. For example, the display 830 may be a speaker or a braille display. While the display 830 is illustrated as a peripheral component in computer system 800, it may also be an integral part of computing device 802.

An input device 832 is also connected, directly or indirectly, to the bus 804. The input device is any input device suitable for enabling a user or system to control the operation of computing device 802. In some embodiments, the input device 832 contains a number of keys, buttons and/or switches, e.g. a keyboard, a gamepad or a measurement process control panel. In other embodiments, the input device 832 is a touch screen integrated with the display 820. The input device 832 may also be a cursor controller, such as a mouse, trackball or trackpad. While the input device 832 is illustrated as a peripheral component in computer system 800, it may also be an integral part of computing device 802.

### MODIFICATIONS

It will be appreciated that various modifications may be made to the embodiments hereinbefore described. Such modifications may involve equivalent and other features which are already known in the design, manufacture and use of measurement process optimisers and component parts thereof and which may be used instead of or in addition to features already described herein. Features of one embodiment may be replaced or supplemented by features of another embodiment.

The configuration and/or implementation of the generated predictors, e.g. the process whereby a posterior distribution is derived, may differ.

Although claims have been formulated in this application to particular combinations of features, it should be understood that the scope of the disclosure of the present invention also includes any novel features or any novel combination of features disclosed herein either explicitly or implicitly or any generalization thereof, whether or not it relates to the same invention as presently claimed in any claim and whether or not it mitigates any or all of the same technical problems as does the present invention. The applicants hereby give notice that new claims may be formulated to such features and/or combinations of such features during the prosecution of the present application or of any further application derived therefrom.

1. A computer implemented method for optimising measurement processes comprising:
receiving one or more parameters for configuring a plurality of measurement processes, wherein the plurality of measurement processes is configured to measure one or more latent variables;
receiving one or more results, wherein each of the results is obtained using at least one of the plurality of measurement processes and the at least one of the plurality of measurement processes are configured using at least one of the received parameters;
generating a predictor, wherein the predictor is usable to provide an estimate of the value of an objective function for a first plurality of unsampled parameter values and is usable to provide an uncertainty value for each of the plurality of unsampled parameter values, wherein generating the predictor comprises:
determining the value of the objective function for the one or more parameters in dependence on the one or more results;
determining one or more updated parameters using the predictor;
receiving one or more further results, wherein each of the further results is obtained using at least one of the plurality of measurement processes and the at least one of the plurality of measurement processes are configured using at least one of the one or more updated parameters;
generating an updated predictor, wherein the updated predictor provides an estimate of the value of an objective function for a second plurality of unsampled parameter values and provides an uncertainty value for each of the plurality of unsampled parameter values, wherein generating the updated predictor comprises:
determining the value of the objective function for the one or more updated parameters in dependence on the one or more further results;
determining one or more enhanced parameters using the updated predictor, wherein the value of the objective function for the one or more parameters in dependence on one or more enhanced results obtained when the measurement processes are configured using the one or more enhanced parameters, is greater than or less than, dependent on the definition of the objective function, the value of the objective function for the one or more parameters.

2. The method of claim 1, wherein the objective function is a measure of the precision of the measurements of the one or more latent variables provided by the plurality of measurement processes.

3. The method of claim 1, further comprising storing the one or more enhanced parameters.

4. The method of claim 1, further comprising performing at least one of the plurality of measurement processes, wherein the measurement processes are configured using the one or more enhanced parameters.

5. The method of claim 1, further comprising:
executing one or more computer programs for performing the plurality of measurement processes;
setting one or more variables of the one or more computer programs based on the one or more parameters; and
setting the one or more variables of the one or more computer programs based on the one or more enhanced parameters,
wherein the results and the further results are received from the one or more computer programs.

6. The method of claim 1, wherein at least one of the plurality of measurement processes comprises measuring the reaction time of a subject.

7. The method of claim 1, wherein the one or more latent variables measure quantifiable aspects of neurocognitive function.

8. The method of claim 7, wherein the aspects of neurocognitive function are each associated with one or more brain networks and/or one or more brain regions.

9. The method of claim 1, wherein the acquisition function is an expected improvement function.

10. The method of claim 1, wherein determining the predictor comprises performing Gaussian process regression and determining the updated predictor comprises performing Gaussian process regression.

11. The method of claim 1, wherein determining the value of the objective function comprises determining the difference between a cross-correlation matrix for the results and a target cross-correlation matrix.

12. The method of claim 1, wherein the plurality of measurement processes are selected from a greater plurality of measurement processes.

13. The method of claim 12, further comprising:
selecting the plurality of measurement processes from the greater plurality of measurement processes.

14. The method of claim 13, wherein selecting the plurality of measurement processes comprises:
receiving initial results for the greater plurality of measurement processes; and
identifying a subset of the greater plurality of measurement processes based on the received initial results, wherein the identified subset is adapted for discretising the measurements of the latent variables.

15. The method of claim 15, wherein identifying the subset comprises performing factor analysis on the received initial results.

16. (canceled)

17. A computer-readable medium comprising instructions which, when executed by a computer, cause the computer to carry out the method claim 1.

18. A computer system comprising one or more processors operatively coupled to one or more memories, wherein the one or more memories store executable instructions which, when executed by the one or more processors, cause the computer system to carry out the method of claim 1.
